(19) United States
(12) Patent Application Publication
(10) Pub. No.: US 2003/0138533 A1
(43) Pub. Date: Jul. 24, 2003

(54) Food and beverage perservative

(75) Inventor: Paul L. Simmons, Pinellas Park, FL (US)

Correspondence Address: KRAMER LEVIN NAFTALIS & FRANKEL LLP, INTELLECTUAL PROPERTY DEPARTMENT, 919 THIRD AVENUE, NEW YORK, NY, 10022, US

(73) Assignee: Nutraceutical Clinical Laboratories International, Inc.

(21) Appl. No.: 10/217,310

(22) Filed: Aug. 12, 2002

Related U.S. Application Data
(63) Continuation-in-part of application No. 10/132,019, filed on Apr. 24, 2002.
(60) Provisional application No. 60/350,792, filed on Jan. 22, 2002.

(51) Int. Cl. A61K 31/717; A23L 3/34

(52) U.S. Cl. 426/321; 514/57

(57) ABSTRACT

Preservative compositions which include vegetable cellulose fiber such that the composition has a pH of at least about 6.6 are provided. Methods for manufacturing such preservative compositions are also provided.

### CROSS-REFERENCE APPLICATIONS

This application is a continuation-in-part of U.S. application Ser. No. 10/132,019, filed Apr. 24, 2002, which claims benefit of U.S. Provisional Application Ser. No. 60/350,792, filed Jan. 22, 2002.

### FIELD OF THE INVENTION

The present invention relates to preservatives formulated to reduce growth of microbial contaminants such as bacteria and spores that cause spoilage of food, beverages and other goods.

### BACKGROUND OF THE INVENTION

Bacteria and other microbial organisms cause food and beverage as well as other products to spoil thereby reducing the shelf life or useful life of such products or goods. Thus, numerous efforts have been made to reduce the deleterious effects of microbial contaminants in products and materials. Previous food and beverage preservative compositions include non-food grade ingredients such as wax paper used as a source of cellulose fiber. Unfortunately, wax paper generally contains bleaches and whiteners that have been shown to be potentially toxic by various LD 50 toxicity tests. These preservative compositions also should be maintained at a pH level of 6.5 or less to prevent the paper from spoiling and to remain effective.

Accordingly, a great need exists for non-toxic, pH-neutral preservative compositions that are capable of reducing microbial contamination and concomitant spoilage of food, beverages and other goods.

### SUMMARY OF THE INVENTION

The present invention relates to preservative compositions formulated to reduce growth of contaminants such as bacteria, mold and spores that cause spoilage of food, beverages and other goods. In contrast to previous preservative compositions, the preservative compositions of the present invention, which include vegetable cellulose fiber, are stable at a substantially neutral pH. Thus, these compositions have advantages over preservatives that must be maintained at a pH below about 6.5. For example, since compositions according to the present invention avoid the use of toxic chemicals, there are fewer regulatory limitations for the use of such compositions in food or beverages. Furthermore, without the problem of toxicity, concentrations can be increased as much as 300% or more to accommodate the amount of liquid or lack thereof in a particular product. In addition, since these compositions are stable at a substantially neutral pH, flavors or fragrances are not required.

Thus, the present invention provides substantially neutral preservative compositions comprising vegetable cellulose fiber, and various combinations of wax such as beeswax, emulsifiers such as soy lecithin, potassium sorbate and water that can be used in products such as foods and beverages.

The present invention also provides methods of manufacturing such preservative compositions. In one embodiment, a method of this invention includes the steps of (1) heating water; (2) adding an emulsifier such as soy lecithin and/or wax such as beeswax to the heated water and mixing; (3) adding vegetable cellulose fiber to the mixture and mixing further; (4) optionally adding potassium sorbate to the mixture; and (5) optionally filtering the mixture.

### DETAILED DESCRIPTION OF THE INVENTION

The present invention provides substantially neutral pH preservative compositions formulated to reduce growth of microbial contaminants such as bacteria, mold and spores that cause spoilage in goods such as foods and beverages.

The preservative compositions of the present invention comprise cellulose fiber and various combinations of wax such as beeswax, an emulsifier such as soy lecithin, potassium sorbate, and water. Such compositions have a substantially neutral pH. As used herein, “substantially neutral pH” means a pH above 6.5 up to about 9.0. Accordingly, as used herein “substantially neutral pH” includes a pH range from slightly acidic to moderately basic. In preferred embodiments the preservative composition is in the form of a viscous liquid.

As used herein, unless otherwise stated, percentage amounts of an ingredient are by total weight of the composition in which that ingredient is included.

The cellulose fiber used in the present invention may include, for example, vegetable, acetyl, hydroxyl or powdered cellulose fiber. The preferred cellulose fiber is a vegetable cellulose fiber. Compositions of the present invention may include vegetable cellulose fiber from about 0.005 to about 0.5 percent, preferably from about 0.05 to about 0.15 percent, and more preferably about 0.10 percent, all by weight of the composition. The vegetable cellulose fiber used in the present invention may be any size, but is preferably less than about 40 microns.

Preservative compositions of the present invention also may include wax material such as beeswax or paraffin. The preferred wax material is beeswax. Wax material such as beeswax provides a coating on the cellulose fibers. This coating deprives bacteria and/or other organisms of oxygen thereby preventing reproduction by oxygen-dependent organisms. Beeswax may comprise from about 0.005 to about 0.5 percent, preferably from about 0.05 to about 0.1 percent, and more preferably about 0.067 percent, all by weight of the composition. The ratio of vegetable cellulose fiber to beeswax may be from about 3:1 to about 1:3, but is preferably from about 3:1 to about 1: 1, and is more preferably from about 2:1 to about 1:1.

The preservative composition may also include an emulsifier. Examples of emulsifiers include soy lecithin, diacetyl tartaric acids, and esters of monoglycerides (e.g., polyoxyethylene sorbitan esters, mono- and diglycerides and sorbitan esters). A preferred emulsifier is soy lecithin. Soy lecithin may comprise from about 0.00001 to about 0.5 percent, preferably from about 0.00005 to about 0.001 percent, and more preferably about 0.0001 percent, all by weight of the composition.

The preservative composition may also include mold inhibitors. Mold inhibitors may include potassium sorbate, sodium benzoate, calcium propionate, and sorbic acid. Potassium sorbate is a preferred mold inhibitor. In embodiments of the present invention, potassium sorbate may comprise from about 0.05 to about 10 percent, preferably from about 1.0 to about 5.0 percent, and more preferably from about 2.0 to about 4.0 percent, all by weight of the composition.

Compositions of the present invention also may include other excipients.

The balance of the composition may comprise water. The water is preferably purified and preferably has a bacterial colony count of less than about 100 colonies per ml. The amount of water used may be varied based on the desired concentration of the preservative.

The pH of these preservative compositions is above 6.5, is preferably above about 7.0 and more preferably is from at least about 7.0 to about 7.8.

This particular combination of food grade ingredients permits the composition to be altered or changed depending of the food, beverage or other product to be preserved while adjusting the pH range to suit the food, beverage or other product.

The present invention also provides methods of making the preservatives described herein. Such methods may include the steps of (1) heating water; (2) adding wax to the heated water and mixing; (3) optionally adding an emulsifier and mixing; (4) adding vegetable cellulose fiber to the mixture and mixing further; (5) optionally adding potassium sorbate to the mixture; (6) optionally filtering the mixture; and (7) optionally cooling the filtrate.

In examples of embodiments of the present invention, the water may be heated to between about 110° to about 272° F., preferably to between about 160° to about 220° F., and more preferably to between about 180° to about 200° F. An emulsifier such as soy lecithin and a wax such as beeswax may be added to the heated water followed by agitation or mixing until a substantially homogenous solution is achieved. This step may be accomplished usually in about 5 minutes, although longer or shorter time may be needed. The vegetable cellulose fiber then may be added and agitated or mixed until forming a substantially homogenous solution. This step usually takes about an additional 5 minutes, although longer or shorter time may be needed. Finally, potassium sorbate may be added to the mixture. The liquid mixture may then be pumped through filters at a pressure sufficient to effectively collect the preservative composition. The filtering pressure may be from about 5 to about 100 psi, preferably is from about 10 to about 40 psi, and more preferably is about 30 psi. The preservative composition is then cooled before use. Preferably the composition is cooled to at least about 95° F.

It should be noted that the order of steps recited above in embodiments of the present invention may be varied to produce compositions according to the present invention. For example, the vegetable cellulose fiber may be added to the heated water prior to adding soy lecithin and/or beeswax. Also, the vegetable cellulose, soy lecithin or other emulsifier and/or wax such as beeswax may be added to the water prior to heating the water.

Preservative compositions according to the present invention may by applied to a product in a number of ways. For example, such compositions may be sprayed, injected, dipped or poured directly onto products. Alternatively, preservative compositions may be frozen and products may be placed in contact with the frozen preservative compositions. Further, preservative compositions may be spray dried, freeze-dried and/or powdered and then applied to products. Preservative compositions may be added to a finished product or may be added at any step in the production processes of a product.

Alternatively, the ingredients of the preservative of the invention as described above can be individually or collectively added to the final product or to what becomes the final product, or in a process of making the final product, either separately or all together at once.

In addition, such preservative compositions may be added to consumer products including, but not limited to cosmetic and personal care products, cleaning agent products, and other products.

Cosmetic and personal care products may include: pigmentation and sun care products; diaper, baby wipe and hand wipe, baby powder and body powder and diaper rash products; nursing pads (for bras); makeup products; tampon, maxipad and pantiliner products; acne prevention and treatment products; facial cleansing, toning and exfoliating products and makeup removal products; facial moisturizing, anti-wrinkle, eye treatment, hand lotion and body lotion products; foot care products; anti-itch products; anti-bacterial, antiseptic, antibiotic and first aid products; bath and shower soap in bar, liquid and gel form and bath salt products; shampoo and hair detangling products; hair mousse, hair gel and hair spray products; anti-perspirant and deodorant products in powder, creme, roll-on, aerosol and stick form; aftershave and shaving lotion products; shaving products in creme, gel, powder and soap forms; depilatory, epilatory and hair bleaching products in creme, wax and powder forms; toothpaste products; mouthwash and mouth rinse products; wig and toupee powder products; shoulder pads; freckle coating products, eye drop products; and contact lens treatment products.

Cleaning agent products may include: laundry detergents, stain removers, and fabric softening products, dish washing products, air fresheners, deodorizing products, bathroom tissues, facial tissues, paper towels, napkin products, cotton swabs, handiwipes, scouring and sponge products, oven cleaning products, toilet cleaning products, tub and shower cleaning products, carpet cleaning products, all purpose cleaning products, and jewelry and metal cleaning products.

In addition to the products listed above, preservatives according to the present invention may be added to or included in the following household products: dust filters, wall paint/wallpaper, toilet seat covers, mold remover, ceramic/bathroom tile laminates, water filters, mattress fillers, cleaning agents for solariums and sun beds, toilet brushes, pet litter, and cutting boards to make these products highly resistant to microbial contamination.

The present invention also provides novel products useful in the animal care and veterinary fields. Preservative compositions according to the present invention may also be added to or included, for example, in the following animal/veterinary products to make these products highly resistant to microbial contamination: sand for bird cages, kitty litter, flea powder, and dry shampoos for animals.

Preservative compositions according to the present invention may also be added to or included in the following products: fungicide/pesticide for agriculture, marine antifoulant, coating for glass and cleaners for industrial food and beverage containers, concrete, ceramics, and tile, to name a few, to make these products highly resistant to microbial contamination.

### EXAMPLE

The composition as shown in Table 1 was used to demonstrate the preservative properties of an exemplary embodiment of the present invention:

TABLE 1

| | |
|---|---|
| vegetable cellulose fiber | 0.1000% |
| beeswax | 0.0670% |
| soy lecithin | 0.0001% |
| potassium sorbate | 3.0000% |
| water | 96.833% |

The test sample preservative composition as shown in Table 1 was added to white pan bread mix in the final minute of mixing after yeast and salt had been blended into the mixture. The preservative composition comprised about 3.5% of the total weight of the mix. A control sample containing 0.27% calcium propionate was added to another identical sample of white pan bread mix. The control sample molded in 12 days. The test sample remained free of any sign of mold growth during a 90 day test period. Both the test sample and the control sample were packaged and stored in the same conditions throughout the test period.

What is claimed is:

1. A food and beverage preservative composition comprising from about 0.005 to about 0.5 percent vegetable cellulose fiber and from about 0.005 to about 0.5 percent wax, all by weight.

2. The preservative composition of claim 1, wherein the wax comprises beeswax.

3. The preservative composition of claim 1, further comprising an emulsifier.

4. The preservative composition of claim 3, wherein the emulsifier is selected from the group consisting of soy lecithin, diacetyl tartaric acids, and esters of monoglycerides.

5. The preservative composition of claim 4, wherein the emulsifier comprises soy lecithin.

6. The preservative composition of claim 5, wherein the wax comprises beeswax.

7. The preservative composition of claim 6, comprising from about 0.005 to about 0.5 percent vegetable cellulose fiber, from about 0.005 to about 0.5 percent beeswax, and from about 0.00001 to about 0.5 percent soy lecithin, all by weight.

8. The preservative composition of claim 7, wherein said composition comprises about 0.05 to about 0.15 percent vegetable cellulose fiber, from about 0.05 to about 0.1 percent beeswax, and from 0.00005 to about 0.001 soy lecithin, all by weight.

9. The preservative composition of claim 8, wherein said composition comprises about 0.01 percent vegetable cellulose fiber, about 0.067 percent beeswax and about 0.0001 or less percent soy lecithin, all by weight.

10. The preservative composition of claim 7, further comprising potassium sorbate from about 0.05 to about 10 percent by weight.

11. The preservative composition of claim 10, comprising potassium sorbate from about 1.0 to about 5.0 percent by weight.

12. The preservative composition of claim 11, comprising potassium sorbate from about 2.0 to about 4.0 percent by weight.

13. The preservative composition of claim 7, wherein the pH of said composition is at least about 6.6.

14. The preservative of claim 7, wherein the pH of said composition is at least about 7.0.

15. A method of manufacturing a food and beverage preservative composition comprising:
(1) heating water to between about 160° and about 220° F.; (2) adding wax and mixing to a substantially homogenous solution; (3) adding vegetable cellulose fiber and mixing to a substantially homogenous solution; and (4) adjusting the pH to at least about 6.6.

16. The method of claim 15, further comprising adding an emulsifier and mixing to a substantially homogenous solution.

17. The method of claim 15, wherein the wax comprises beeswax.

18. The method of claim 16, wherein the wax comprises beeswax.

19. The method claim 15, further including the step of filtering said composition at a pressure no greater than about 40 psi.

20. The method of claim 15, further including the step of cooling said composition to less than about 95° F.

21. The method of claim 16, wherein said emulsifier comprises soy lecithin.

22. The method of claim 21, wherein said preservative composition comprises from about 0.005 to about 0.5 percent vegetable cellulose fiber, from about 0.005 to about 0.5 percent beeswax, and from about 0.00001 to about 0.5 percent soy lecithin, all by weight.

23. The method of claim 22, wherein said preservative composition comprises from about 0.05 to about 0.15 percent vegetable cellulose fiber, from about 0.05 to about 0.1 percent beeswax and from about 0.00005 to about 0.002 percent soy lecithin, all by weight:

24. The method of claim 23, wherein said preservative composition comprises about 0.01 percent vegetable cellulose fiber; about 0.067 percent beeswax and about 0.0001 or less percent soy lecithin, all by weight.

25. The method of claim 15, wherein the composition further comprises from about 0.5 to about 10 percent potassium sorbate, by weight.

26. The method of claim 25, wherein the composition comprises from about 1 to about 5 percent potassium sorbate, by weight.

27. A food or beverage product comprising the composition of any of claims 1-12.

28. A preserved food or beverage product comprising from about 0.0005 to about 0.5 percent vegetable cellulose fiber and from about 0.0005 to about 0.5 percent wax, all by weight of the product.

29. A method of preserving a food or beverage product comprising adding from about 0.0005 to about 0.5 percent vegetable cellulose fiber and from about 0.0005 to about 0.5 percent wax, all by weight of the product

30. A consumer product comprising from about 0.0005 to about 0.5 percent vegetable cellulose fiber and from about 0.0005 to about 0.5 percent wax, all by weight of the product.

31. A method of preserving a consumer product comprising adding from about 0.0005 to about 0.5 percent vegetable cellulose fiber and from about 0.0005 to about 0.5 percent wax, all by weight of the product.
